(19) United States
(12) Patent Application Publication
(10) Pub. No.: US 2023/0241229 A1
(43) Pub. Date: Aug. 3, 2023

(54) A SAPOGENIN POLYETHYLENE GLYCOL ESTER COMPLEX OF FORMULA-I, A COMPOSITION, PROCESS OF PREPARATION, AND APPLICATIONS THEREOF

(71) Applicant: PHYTOSOL INDIA PRIVATE LIMITED, Bangalore, Kamataka (IN)

(72) Inventors: Velmurugan SHANMUGHAM, Bangalore (IN); Suresh NARAYAN, Bangalore (IN)

(21) Appl. No.: 17/918,912

(22) PCT Filed: Apr. 16, 2021
(86) PCT No.: PCT/IB2021/053152

§ 371 Date: Oct. 14, 2022

(30) Foreign Application Priority Data
Apr. 16, 2020 (IN) 202041016518

(51) Int. Cl. A61K 47/60; A61K 47/54; A61K 31/12; A61K 9/48; A61K 31/352; A61K 9/00; A61K 9/16; A61K 9/14; A61K 31/015; A61K 8/63; A61Q 19/00; A23L 2/52; A23G 3/36

(52) U.S. Cl. CPC A61K 47/60; A61K 47/542; A61K 31/12; A61K 9/4825; A61K 31/352; A61K 9/0095; A61K 9/1664; A61K 9/1617; A61K 9/1611; A61K 9/141; A61K 31/015; A61K 8/63; A61Q 19/00; A23L 2/52; A23G 3/36; A61K 2800/10

(57) ABSTRACT

The present disclosure relates to field of Pharmaceutical Chemistry. The present disclosure particularly relates to Compound of Formula-I and process of preparation of the said Compound of Formula-I. The present disclosure further relates to Composition comprising the Compound of Formula-I and active ingredients, such as active pharmaceutical ingredient, dietary ingredients and cosmetic ingredients. The said composition possesses improved solubility and improved bioavailability for the active ingredients such as active pharmaceutical ingredient, dietary ingredients and cosmetic ingredients.

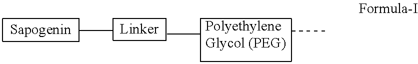

### CROSS-REFERENCE TO RELATED APPLICATIONS

This application is a § 371 National Phase entry of international Application No. PCT/IB2021/053152, filed Apr. 16, 2021, which claims the benefit of priority to Indian Patent Application Number 202041016518, filed Apr. 16, 2020, the entire contents of each of which are hereby incorporated by reference.

### TECHNICAL FIELD

The present disclosure particularly relates to Compound of Formula-I representing Sapogenin Polyethylene Glycol ester and process of preparation of said Compound of Formula-I. The present disclosure further relates to composition comprising the said Compound of formula-I and active ingredients, such as active pharmaceutical ingredient, dietary ingredients and cosmetic ingredients. The said composition possesses improved solubility and improved bioavailability for the active ingredients such as active pharmaceutical ingredient, dietary ingredients and cosmetic ingredients.

### BACKGROUND OF THE DISCLOSURE

In general, ingredients such as Drugs, Biologicals, Dietary & Cosmetic ingredients are insoluble in water. This hydrophobic property often makes it difficult to formulate it so that it exhibits a satisfactory bioavailability and systemic availability. Poorly water-soluble ingredients lead to less absorption in the body and thereby poor bioavailability.

The limited solubility prohibits the ingredient when formulating the oral dosage, in particular. Poorly water-soluble Drugs, biologicals, Dietary and Cosmetic ingredients have less absorption and low availability in the target, which leads to less therapeutic effects. In order to achieve the desired therapeutic effect for poorly water-soluble ingredients, a high dose is always must but that leads to undesired side effects.

The Biopharmaceutics Classification System (BCS) is a system developed to differentiate drugs based on their solubility and permeability and can be used for predicting intestinal absorption of the drug. The BCS system categorizes drugs into four classes based on their solubility and intestinal permeability: class I drugs have high permeability and high solubility; class II drugs have high permeability and low solubility; class III drugs have low permeability and high solubility; and class IV drugs have low permeability and low solubility. Accordingly, formulation of BCS class II and IV drugs (i.e., poorly soluble drugs) presents significant challenges in the pharmaceutical industry.

For instance, Curcumin (diferuloylmethane) is a yellow pigment present in the spice turmeric (Curcuma longa) that has been associated with antioxidant, anti-inflammatory, anticancer, antiviral, and antibacterial activities. The optimal health benefits of curcumin are limited by its low solubility in water and corresponding poor intestinal absorption. Similarly, there are numerous active ingredients which currently have limited applications due to its low solubility and reduced bioavailability.

Accordingly, there remains a need in the art for improved formulations for poorly soluble Drugs, active ingredients, Biologicals, Dietary & Cosmetic ingredients. Present invention aims to fulfill this need and provides further related advantages.

### SUMMARY OF THE DISCLOSURE

Accordingly, the present disclosure relates to Compound of Formula-I

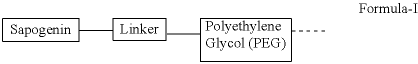

The present disclosure further relates to method of preparing the compound of formula-I, said method comprises—mixing the sapogenin, the carboxylic acid/dicarboxylic acid/derivative thereof, the PEG and solvent-A to obtain a mixture; coiling the mixture, followed by removing solvent-A; adding solvent-B followed by washing with solution and cooling the mixture, followed by washing with solvent-C and drying to obtain the compound.

The present disclosure further relates to a composition comprising the Compound of Formula-I and active ingredient selected from a group comprising active pharmaceutical ingredient, dietary ingredient, cosmetic ingredient and a combination thereof, optionally along with excipient including but not limited to surfactant polymers and dispersing agents.

The present disclosure further relates to a method of preparing the composition described above, said method comprises—mixing the compound of formula I and the active ingredient to obtain a mixture; and homogenizing the mixture to obtain the composition.

### BRIEF DESCRIPTION OF THE ACCOMPANYING FIGURES

FIG. 1: Illustrates the dissolution of the curcumin from the composition of the present disclosure.

FIG. 2: Release of curcumin from the composition of the present disclosure in comparison to the release of curcumin from the compositions comprising commercially available surfactants, respectively.

### DETAILED DESCRIPTION OF THE DISCLOSURE

In the following description, certain specific details are set forth in order to provide a thorough understanding of various embodiments of the invention. However, one skilled in the art will understand that the invention may be practiced without these details.

Unless the context requires otherwise, throughout the present specification, the word “comprise” and variations thereof, such as, “comprises” and “comprising” are to be construed in an open, inclusive sense, that is as “including, but not limited to”, without deviating from the scope that the present invention intends to describe/cover.

Reference throughout this specification to “one embodiment” or “an embodiment” means that a particular feature, structure or characteristic described in connection with the embodiment is included in at least one embodiment of the present invention.

Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments active pharmaceutical ingredient can be
a) A substance recognized by an official pharmacopoeia or formulary.
b) A substance intended for use in the diagnosis, cure, mitigation, treatment, or prevention of disease.
c) A substance (other than food) intended to affect the structure or any function of the body.
d) A substance intended for use as a component of a medicine but not a device or a component, part or accessory of a device.
e) Biological products are included within this definition and are generally covered by the same laws and regulations, but differences exist regarding their manufacturing processes (chemical process versus biological process.)
f) Pharmaceutically acceptable salt of respective drugs

Biological products include a wide range of products such as vaccines, blood and blood components, allergenics, somatic cells, gene therapy, tissues, and recombinant therapeutic proteins. Biologics can be composed of sugars, proteins, or nucleic acids or complex combinations of these substances, or may be living entities such as cells and tissues.

Biologics are isolated from a variety of natural sources viz human, animal, or microorganism and produced by biotechnology methods and also includes Gene-based and cellular biological products.

Dietary ingredients are vitamin; mineral; herb or other botanical; amino acid; dietary substance for use by man to supplement the diet by increasing the total dietary intake; or a concentrate, metabolite, constituent, extract, or combination of the preceding substances.
a) vitamin and mineral products
b) “botanical” or herbal products—These come in many forms and may include plant materials, algae, macroscopic fungi, or a combination of these materials.
c) amino acid products—Amino acids are known as the building blocks of proteins and play a role in metabolism.
d) enzyme supplements—Enzymes are complex proteins that speed up biochemical reactions.

The dietary ingredients can be used in Ayurvedic formulations, Functional food beverages, Health Functional foods, Food for Specific health uses, Dietary supplements, Natural health products, Food supplements, Novel foods, Traditional herbal medicinal products.

Cosmetic ingredients are “articles intended to be rubbed, poured, sprinkled, or sprayed on, introduced into, or otherwise applied to the human body for cleansing, beautifying, promoting attractiveness, or altering the appearance.” Included in this definition are products such as skin moisturizers, perfumes, lipsticks, fingernail polishes, eye and facial makeup preparations, shampoos, permanent waves, hair colors, toothpastes, and deodorants, as well as any material intended for use as a component of a cosmetic product.

The present disclosure relates to Compound of Formula-I

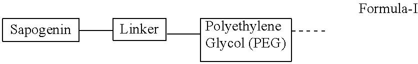

In an embodiment, the Sapogenin is selected from a group comprising Diosgenin, Solasodin, Tomatidine, 11-Ketotigogenin, Hecogenin, Laxogenin, Smilagenin, Sarsasapogenin, Tigogenin, Epismilagenin, Yamogenin and any combination thereof.

In an embodiment source of the Sapogenin is naturally derived from plant, animal sources and synthetic and semi synthetic derived from animal and plant sources.

In another embodiment the sapogenin is commercially available.

In an embodiment, the linker is a carboxylic/dicarboxylic and/or its derivatives selected from the group consisting of Succinate, adipate, glutarate, pimelate malonate and Sebecate.

In an embodiment, the Polyethylene glycol (PEG) is selected from a group comprising PEG 200, PEG 400, PEG 600, PEG 800, PEG 1000, PEG 2000 and any combination thereof.

In an embodiment, the compound according to the said Compound of Formula-I is selected from a group comprising Diosgenin Polyethylene glycol succinate, Diosgenin Polyethylene Glycol Adipate, Diosgenin Polyethylene Glycol Glutarate, Diosgenin Polyethylene Glycol Pimelate, Diosgenin Polyethylene Glycol Malonate, Diosgenin Polyethylene Glycol Sebecate, Tomatidine Polyethylene glycol succinate, Tomatidine Polyethylene Glycol Adipate, Tomatidine Polyethylene Glycol Glutarate, Tomatidine Polyethylene Glycol Pimelate, Tomatidine Polyethylene Glycol Malonate, Tomatidine Polyethylene Glycol Sebecate, 11-Ketotigogenin Polyethylene glycol succinate, 11-Ketotigogenin Polyethylene Glycol Adipate, 11-Ketotigogenin Polyethylene Glycol Glutarate, 11-Ketotigogenin Polyethylene Glycol Pimelate, 11-Ketotigogenin Polyethylene Glycol Malonate, 11-Ketotigogenin Polyethylene Glycol Sebecate, Hecogenin Polyethylene glycol succinate, Hecogenin Polyethylene Glycol Adipate, Hecogenin Polyethylene Glycol Glutarate, Hecogenin Polyethylene Glycol Pimelate, Hecogenin Polyethylene Glycol Pimelate, Hecogenin Polyethylene Glycol Malonate, Hecogenin Polyethylene Glycol Sebecate, Laxogenin Polyethylene glycol succinate, Laxogenin Polyethylene Glycol Adipate, Laxogenin Polyethylene Glycol Glutarate, Laxogenin Polyethylene Glycol Pimelate, Laxogenin Polyethylene Glycol Malonate, Laxogenin Polyethylene Glycol Sebecate, Smilagenin Polyethylene glycol succinate, Smilagenin Polyethylene Glycol Adipate, Smilagenin Polyethylene Glycol Glutarate, Smilagenin polyethylene Glycol Pimelate, Smilagenin Polyethylene Glycol Malonate, Sarsasapogenin Polyethylene glycol succinate, Sarsasapogenin Polyethylene Glycol Adipate, Sarsasapogenin Polyethylene Glycol Glutarate, Sarsasapogenin Polyethylene Glycol Pimelate, Sarsasapogenin Polyethylene Glycol Malonate Sarsasapogenin Polyethylene Glycol Sebecate, Tigogenin Polyethylene glycol succinate, Tigogenin Polyethylene Glycol Adipate, Tigogenin Polyethylene Glycol Glutarate, Tigogenin Polyethylene Glycol Pimelate, Tigogenin Polyethylene Glycol Malonate, Tigogenin Polyethylene Glycol Sebecate, Epismilagenin Polyethylene Glycol succinate, Epismilagenin Polyethylene Glycol Adipate, Epismilagenin Polyethylene Glycol Glutarate, Epismilagenin Polyethylene Glycol Pimelate, Epismilagenin Polyethylene Glycol Malonate, Epismilagenin Polyethylene Glycol Sebecate, Yamogenin Polyethylene Glycol succinate, Yamogenin Polyethylene Glycol Adipate, Yamogenin Polyethylene Glycol Glutarate, Yamogenin Polyethylene Glycol Pimelate, Yamogenin Polyethylene Glycol Malonate and Yamogenin Polyethylene Glycol Sebecate.

In an exemplary embodiment, the compound of formula-I is Diosgenin Polyethylene glycol succinate having the following molecular structure—

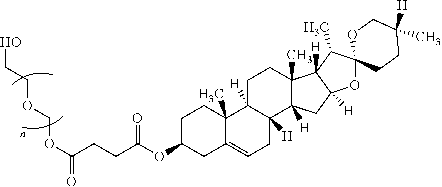

The present disclosure further relates to process of preparing the said Compound of Formula-I.

In an embodiment, the process of preparing the said Compound of Formula-I comprises converting the sapogenin into carboxylic ester derivative and further converting into the compound of formula-I in a sequential manner employing minimum number of steps.

In an embodiment of the present disclosure, the method of preparing the Compound of Formula I comprising steps of: (a) reacting a mixture of the Sapogenin, the Dicarboxylic and the PEG in an organic solvent to obtain a reaction mixture; (b) cooling the reaction mixture of step (a); (c) filtering the cooled reaction mixture of step (b) to obtain a filtrate; and (d) washing the filtrate with brine solution to obtain the final compound.

In another embodiment of the present disclosure, the method of preparing the compound of formula-I comprises—
a) heating solvent-A, followed by adding sapogenin, dicarboxylic acid and stirring, followed by heating to obtain a mixture;
b) cooling the mixture and adding PEG, followed by heating;
c) distilling solvent-A and adding solvent-B, followed by washing with a solution
d) cooling the mixture of step c), followed by washing with solvent-C and drying to obtain the compound of formula-I.

In an embodiment, in the method described above, the mixture of step (a) further comprises a base selected from the group consisting of DMAP, DIPEA, pyridine, and TEA. In some cases, the base is present in a catalytic amount.

In an embodiment, in the method described above, the mixture of step (b) further comprises an acid selected from a group comprising sulfuric acid, p-toluene Sulfonic acid, or methane sulfonic acid. In some embodiments, the acid is present in a catalytic amount.

In an embodiment, the organic solvent is selected from the group comprising Toluene, Ethyl acetate, Methanol, Ethanol Isopropyl alcohol and any combination thereof.

In an embodiment, in the above described methods, the solvent-A is selected from a group comprising Toluene, N N Dimethylformamide and Xylenes; and the solvent-B is selected from a group comprising ethyl acetate, Isopropyl acetate and Isobutyl acetate; and the solvent-C is selected from Petroleum ether with the boiling range of about 40 to 60° C. or about 60 to 80° C.; Diethyl ether, Methyl t-butyl ether.

In an embodiment, in the above described methods, the solvent of step-B is removed after the step of washing. In an embodiment, in the above described methods, the drying is carried out under vacuum.

In an embodiment, in the above described methods, the solution is about 25% to 50% brine solution.

In an embodiment, in the above described methods, in Step A, the base is selected from a group comprising triethylamine (TEA), Trioctyl amine (TOA), tributyl amine (TBA) and Pyridine.

In an embodiment, in the above described methods, the dicarboxylic acid in Step-A, is selected from a group comprising Succinic anhydride, Succinyl dichloride, Succinic dibromide.

In an exemplary embodiment, the method of preparing the Diosgenin Polyethylene Glycol succinate comprises:

Step-A: Toluene is heated to a temperature ranging from about 60° C. to 70° C., followed by adding Diosgenin, Succinic anhydride and DMAP to heated toluene under stirring. The reaction mass was heated to a temperature of about 110° C. and maintained for a duration ranging from about 12 hours to 16 hours. The reaction mass was cooled to a temperature ranging from about 0° C. to 5° C. To the cooled reaction mass, Sulfuric acid and PEG 1000 was added. The reaction mass was further heated to a temperature of about 110° C. and maintained for a duration ranging from about 4 hours to 6 hours.
Step-B: Toluene was distilled off and Ethyl acetate was added and washed with 25% Brine solution for at least 4 times.
Step-C: The product was chilled and washed with Petroleum ether, followed by drying under vacuum to obtain Diosgenin Polyethylene glycol succinate.

In another embodiment of the present disclosure, the method for preparing the compound described above comprises—
a) mixing the sapogenin, the carboxylic acid/dicarboxylic acid/derivative thereof, the PEG and solvent-A to obtain a mixture;
b) cooling the mixture, followed by removing solvent-A;
c) adding solvent-B, followed by washing with solution;
d) cooling the mixture of step c), followed by washing with Solvent-C and drying to obtain the compound.

In an embodiment of the present disclosure, wherein the mixing of step a) comprises—
a) heating solvent-A, followed by adding sapogenin and the carboxylic acid/dicarboxylic acid/derivative and continuing heating; and
b) adding an acid and PEG, followed by heating to obtain a mixture;

In an embodiment of the present disclosure, wherein the mixing of step a) optionally comprises—
a) Mixing sapogenin and solvent-A, followed by adding a base and carboxylic acid/dicarboxylic acid/derivative to obtain mixture-A;
b) Mixing PEG, solvent-A and base to obtain mixture-B; and
c) Mixing the mixture-A and the mixture-B.

In an embodiment of the present disclosure, wherein the solvent-A is selected from a group comprising Toluene, N N Dimethylformamide, Xylene and combinations thereof; wherein the solvent-B is selected from a group comprising ethyl acetate, Isopropyl acetate, Isobutyl acetate and combinations thereof; wherein the solvent-C is selected from a group comprising petroleum ether, diethyl ether, methyl t-butyl ether and combinations thereof; and wherein the solution is brine solution.

In an embodiment of the present disclosure, wherein the mixture in step b) is cooled to a temperature ranging from about 5° C. to 15° c; and wherein the mixture of step d) is cooled to a temperature ranging from about 0° C. to 5° C.

In an embodiment of the present disclosure, wherein the solvent-A is heated to a temperature ranging from about 80° C. to 90° C.; and wherein the heating is continued to a temperature ranging from about 100° C. to 120° C. for a duration ranging from about 16 hours to 18 hours.

In an embodiment of the present disclosure, wherein the heating in step b) is carried. out for a duration ranging from about 100° C. to 120° C. for a duration ranging from about 2 hours to 8 hours.

In an embodiment the compound of formula-I acts as bioavailable enhancers.

In an embodiment, the compound of formula-I can be used to protect the Thermolabile/oxidative degenerative ingredients alone in single or in combination of Carotenoids, lutein, Lycopene, Capsanthin, Kryptoxanthin, Astaxanthin, Fucoxanthin, Zeaxanthin, Kryptoxanthin and the like.

In an embodiment, the compound of formula-I can be used as Enteric coating agent.

In an embodiment the compound of formula-I can be used as Stability enhancers for Food additives.

In an exemplary embodiment, the compound of formula-I, such as Diosgenin polyethylene carboxylic ester is more bioavailable due its increased solubility in water than Diosgenin which is insoluble in water.

In an exemplary embodiment, the compound of formula-I, such as Diosgenin polyethylene carboxylic ester acts as anti-inflammatory agents in arthritis conditions.

In an exemplary embodiment, the compound of formula-I, such as Diosgenin polyethylene carboxylic ester acts as anti-diabetic agent.

In an exemplary embodiment, the compound of formula-I, such as Diosgenin polyethylene carboxylic ester acts as anti-cholesterol agent.

In an exemplary embodiment, the compound of formula-I, such as Diosgenin polyethylene carboxylic ester acts as anti-obesity agents.

In an exemplary embodiment, the compound of formula-I, such as Diosgenin polyethylene carboxylic ester can be used to treat alcoholic and non-alcoholic fatty liver diseases.

In an exemplary embodiment, the compound of formula-I, such as Diosgenin polyethylene carboxylic ester can be used to treat Dementia and improves cognitions.

In an embodiment, the present disclosure further relates to a composition comprising compound of formula-I and active ingredient selected from a group comprising active pharmaceutical ingredient, dietary ingredient and cosmetic ingredient, optionally along with excipient.

In an embodiment, the ratio of active ingredient to compound of formula-I in the composition is ranging from about 1:1 to 1:10.

In another embodiment, the ratio of active ingredient to compound of formula-I in the composition is about 1:1, about 1:2, about 1:3, about 1:4, about 1:5, about 1:6, about 1:7, about 1:8, about 1:9 or about 1:10.

In another embodiment, the active ingredient is present in the composition in a mass percentage ranging from about 1% to 10%.

In another embodiment, the active ingredient is present in the composition in a mass percentage of about 1%, about 2%, about 3%, about 4%, about 5%, about 6%, about 7%, about 8%, about 9% or about 10%.

In an embodiment, the active pharmaceutical ingredient is selected from a group comprising drugs, biologicals, Pesticides and Phytochemicals.

In an embodiment, the drug is BCS class II, BCS Class IV or a combination thereof.

In an embodiment, the drug belonging to BCS class II or BCS IV is selected from a group comprising azathioprine, dapsone, furosemide, mebendazole, ofloxacin, phenazopyridine, sulfamethoxazole, and talinolol. The BCS classification system is well-known to those of ordinary skill in the art and is described in more detail in Takagi T, Ramachandran C, Bermejo M, Yamashita S, Yu LX, Amidon GL. “A provisional biopharmaceutical classification of the top 200 oral drug products in the United States, Great Britain, Spain, and Japan” Mol Pharmaceutics. 2006;3:631-643, the full disclosure of which is hereby incorporated by reference in its entirety.

The term “BCS class II drug” refers to drug substances with high permeability and low solubility. A drug has high permeability when the extent of absorption in humans is determined to be >90% of an administered dose based on mass-balance pharmacokinetics studies, absolute bioavailability studies, or in comparison to an intravenous reference dose. A drug has low solubility when the highest dose strength is not soluble in <250 ml water over a pH range of 1 to 7.5, based on a shake-flask or titration method and analyzed by a validated stability-indicating assay. Non-limiting examples of BCS class II and predicted BCS class II drugs include the following drugs: albendazole, amiodarone, atorvastatin, azithromycin, camptothecin, carbamazepine, carvedilol, chlorpromazine, ciprofloxacin, cisapride, clofazamine, cyclosporine, danazol, diclofenac, diflunisal digoxin, efavirenz, erythromycin, famotidine, fenofibrate, flurbiprofen, gilbenclamide, glipizide, glyburide, griseofulvin, haloperidol, ibuprofen, indinavir, indomethacin, itraconazole, ivermectin, ketoconazole, lansoprazole, lopinavir, lovastatin, mefloquin, nalidixic acid, naproxen, nelfinavir, nevirapine, oxaprozin, phenytoin, piroxicam, praziquantel, raloxifene, rentinol palmitate, rifampin, ritonavir, saquinavir, sirolimus, spironolactone, sulfasalazine, tacrolimus, tamoxifen, terfenadine, and warfarin.

The term “BCS class IV drug” refers to drug substances with low permeability and low solubility. A drug has low permeability when the extent of absorption in humans is determined to be <90%> of an administered dose, based on mass-balance pharmacokinetics studies, absolute bioavailability studies, or in comparison to an intravenous reference dose. A drug has low solubility when the highest dose strength is not soluble in <250 ml water over a pH range of 1 to 7.5, based on a shake-flask or titration method and analyzed by a validated stability-indicating assay. Non-limiting examples of BCS class IV and predicted class IV drugs include the following drugs: acetazolamide, amphotericin, amphotericin B, amuvatinib, chlorothiazide, chlorthalidone, ciprofloxacin, colistin, hydrochlorothiazide, methotrexate, neomycin, nitrofurantoin, and nystatin.

In another embodiment, the active pharmaceutical ingredient is chemotherapeutic agent selected from a group comprising a mitotic inhibitor, alkylating agent, anti-cell cycle inhibitor, enzymes, topoisomerase inhibitor, biological response modifier, anti-hormone, antiangiogenic agent, anti-androgen, platinum coordination complex, substituted urea, methylhydrazine derivative, adrenocortical suppressant, hormone and hormone antagonist, progestin, estrogen, antiestrogen, androgen, and aromatase inhibitor, DNA damaging agent, wherein the DNA damaging agent may be selected, for example, from the group consisting of gamma radiation; platinum, such as cisplatin, carboplatin, satraplatin, and oxaliplatin; topoisomerase I inhibitors, such as camptothecin, irinotecan, and topotecan; and topoisomerase II inhibitors, such as etoposide and teniposide.

In yet another embodiment, active pharmaceutical ingredient is a chemotherapeutic agent selected from a group comprising mitotic inhibitors, alkylating agents, anti-metabolites, cell cycle inhibitors, enzymes, topoisomerase inhibitors such as CAMPTOSAR (irinotecan), biological response modifiers, anti-hormones, antiangiogenic agents such as MMP-2, MMP-9 and COX-2 inhibitors, anti-androgens, platinum coordination complexes (cisplatin, etc.), substituted ureas such as hydroxyurea, methylhydrazine derivatives e.g., procarbazine, adrenocortical suppressants e.g., mitotane or aminoglutethimide, hormone and hormone antagonists such as the adrenocorticosteriods (e.g., prednisone), progestins (e.g., hydroxyprogesterone caproate), estrogens (e.g., diethylstilbesterol), antiestrogens such as tamoxifen, androgens e.g., testosterone propionate, and aromatase inhibitors such as anastrozole, and exemestane, signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, such as HERCEPTIN (Genentech, Inc., South San Francisco, Calif.). EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein, for example erlotinib and imatinib, as CTLA4 (cytotoxic lymphocite antigen 4) antibodies, and other agents capable of blocking CTLA4; and anti-pro liferative agents such as other farnesyl protein transferase inhibitors, for example the farnesyl protein transferase inhibitors.

Examples of DNA-damaging agent is cisplatin, carboplatin, satraplatin, and oxaliplatin; topoisomerase I inhibitors, such as camptothecin, irinotecan, and topotecan; and topoisomerase II inhibitors, such as etoposide and teniposide.

Examples of alkylating agents are, fluorouracil (5-FU) alone or in further combination with leukovorin; other pyrimidine analogs such as UFT, capecitabine, gemcitabine and cytarabine, the alkyl sulfonates, e.g., busulfan (used in the treatment of chronic granulocytic leukemia), improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil (used in the treatment of chronic lymphocytic leukemia, primary macroglobulinemia and non-Hodgkin's lymphoma), cyclophosphamide (used in the treatment of Hodgkin's disease, multiple myeloma, neuroblastoma, breast cancer, ovarian cancer, lung cancer, Wilm's tumor and rhabdomyosarcoma), estramustine, ifosfamide, novembrichin, prednimustine and uracil mustard (used in the treatment of primary thrombocytosis, non-Hodgkin's lymphoma, Hodgkin's disease and ovarian cancer); and. triazines, e.g., dacarbazine (used in the treatment of soft tissue sarcoma).

Examples of antimetabolite chemotherapeutic agents are folic acid analogs, e.g., methotrexate (used in the treatment of acute lymphocytic leukemia, choriocarcinoma, mycosis fungiodes, breast cancer, head and neck cancer and osteogenic sarcoma) and pteropterin; and the purine analogs such as mercaptopurine and thioguanine which find use in the treatment of acute granulocytic, acute lymphocytic and chronic granulocytic leukemias.

Examples of natural product-based chemotherapeutic agents are the vinca alkaloids, e.g., vinblastine (used in the treatment of breast and testicular cancer), vincristine and vindesine; the epipodophyllotoxins, e.g., etoposide and teniposide, both of which are useful in the treatment of testicular cancer and Kaposi's sarcoma; the antibiotic chemotherapeutic agents, e.g., daunorubicin, doxorubicin, epirubicin, mitomycin (used to treat stomach, cervix, colon, breast, bladder and pancreatic cancer), dactinomycin, temozolomide, plicamycin, bleomycin (used in the treatment of skin, esophagus and genitourinary tract cancer); and the enzymatic chemotherapeutic agents such as L-asparaginase.

Examples of EGFR-inhibiting agents are the monoclonal antibodies C225 and anti-EGFR 22 Mab (ImClone Systems, Inc., New York, N.Y.), the compounds ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.), and EGF fusion toxin (Seragen Inc., Hopkinton, Mass).

In an embodiment, the dietary ingredient is phytochemicals selected from a group comprising 3-O-Acetyl-11-keto-beta-boswellic acid, Allicin, Allin, Astaxanthin, Hesperidin, Azadirechtin, beta-carotene, Capsanthin, Carnosic acid, Coenzyme Q-10, Curcuminoids, Ellagic acid, Forskolin, Fucoxanthin, Gingerols, Lutein, Luteolin. Lycopene, Mangiferin, Naringin, Oleanolic acid, Oleuropein, Oxyresveratrol, Piperine, Policosanol, Pterostilbene, Quercetin, Resveratrol, Rosmarinic acid, Rutin, Sesamin, Sesamolin, Silymarin, Tetrahydrocurcuminoids, Ursolic acid, Zeaxanthin Hesperidine and any combination thereof.

In another embodiment, the dietary ingredient is herbal extracts selected from a group comprising Boswellia serrata, Coleus forskholii, Curcuma longa, Withanaia somnifera, Bacopa monnieri, Centella asiatica, Commiphora mukul, Trigonella foenum greacum, Tagetes erecta, Sunflower medium chain triglycerides, Soya medium chain triglycerides, Saw palmetto, Zingiber officinale and any combination thereof.

In another embodiment, the dietary ingredient is Essential oils selected from a group comprising Amaranthus, Black pepper, Cardamom, Cinnamon, Citronella, Clove, Coconut, Coriander, Cumin, Curry leaves, Fennel, Fenugreek, Galangal, Ginger, Lemongrass, Mustard, Nutmeg, Orange, Peppermint, Rosehip, Rosemary, Sage, Spearmint, Star anise, Tea tree, Thyme, Neem, Turmeric, Vetiver.

In another embodiment, the dietary ingredient is Nutritional fine chemicals selected from a group comprising Alpha lipoic acid, Diindolylmethane, Inole-3-carbinol, Ipriflavone, Allyl isothiocynate, Sulforaphane.

In another embodiment the dietary ingredients are Essential fatty acids selected from a group comprising α-Linolenic acid or ALA (18:3n-3), Linoleic acid or LA (18:2n-6), Eicosapentaenoic acid or EPA (20:5n-3), Docosahexaenoic acid or DHA (22:6n-3), gamma-linolenic acid or GLA (18:3n-6), dihomo-gamma-linolenic acid or DGLA (20:3n-6)

In another embodiment the dietary ingredients are Vitamins selected from a group comprising Vitamin A, Vitamin Vitamin D and Vitamin K. Vitamin A comprises of—Retinol, Retinal, Alpha carotene, Beta carotene. Vitamin D comprises of Cholecalciferol (D3), Ergocalciferol (D2). Vitamin E comprises of—Tocopherols, Tocotrienols. Vitamin K comprises of—Phylloquinone and Menaquinones.

In another embodiment, the dietary ingredients are Amino acids selected from a group comprising Alanine, Isoleucine, Leucine, Methionine, Phenylalanine, Valine Proline and Glycine.

In an embodiment, the dietary ingredient are Substances that are added to food to maintain or improve the safety, freshness, aroma, taste, texture, or appearance, color and the like.

In an embodiment, the compound of formula-I in the composition enhances the stability for the ingredients by preventing the ingredients from heat and oxidative degeneration.

In an embodiment, the compound of formula-I in the composition acts as a solubilizer,

The present disclosure further relates to process of preparing the composition.

In an embodiment, the process of preparing the composition comprising steps of: mixing the compound of formula-I and the active ingredient to obtain a mixture; and

homogenizing the mixture to obtain the composition.

In another embodiment, the process of preparing the composition comprising steps of:
Mixing the compound of formula-I and the active ingredient under stirring at a temperature ranging from about 50° C. to 100° C. for a duration of about 1 hour to 3 hours to obtain a mixture;
Homogenizing the mixture at a speed ranging from about 5000 rpm to 10000 rpm for a duration ranging from about 5 minutes to 10 minutes; and
Stirring the homogenized mixture at a temperature of about 25° C. to 50° C. for a duration ranging from about 10 minutes to 15 mintues to obtain the composition.

In an exemplary embodiment, the process of preparing the composition comprising steps of:
Mixing the compound of formula-I and the active pharmaceutical ingredient under stirring at a temperature ranging from about 50° C. to 100° C. for a duration of about 1 hour to 3 hours to obtain the mixture;
Homogenizing the mixture at a speed ranging from about 5000 rpm to 10000 rpm for a duration ranging from about 5 minutes to 10 minutes; and
Stirring the homogenized mixture at a temperature of about 25° C. to 50° C. for a duration ranging from about 10 minutes to 15 minutes to obtain the composition.

In another exemplary embodiment, the process of preparing the composition comprising steps of:
Mixing the compound of formula-I and the dietary ingredient under stirring at a temperature ranging from about 50° C. to 100° C. for a duration of about 1 hour to 3 hours to obtain the mixture;
Homogenizing the mixture at a speed ranging from about 5000 rpm to 10000 rpm for a duration ranging from about 5 minutes to 10 minutes; and
Stirring the homogenized mixture at a temperature of about 25° C. to 50° C. for a duration ranging from about 10 minutes to 15 mintues to obtain the composition.

In another exemplary embodiment, the process of preparing the composition comprising steps of:
Mixing the compound of formula-I and the cosmetic ingredient under stirring at a temperature ranging from about 50° C. to 100° C. for a duration of about 1 hour to 3 hours to obtain the mixture;
Homogenizing the mixture at a speed ranging from about 5000 rpm to 10000 rpm for a duration ranging from about 5 minutes to 10 minutes; and
Stirring the homogenized mixture at a temperature of about 25° C. to 50° C. for a duration ranging from about 10 minutes to 15 mintues to obtain the composition.

In an embodiment of the present disclosure, the composition is formulated into forms selected from a group comprising capsule, tablet, injection, cream, gel, ointment, lotion, solution, emulsion, foam, troche, lozenge, aqueous or oily suspension, patch, dentifrice, spray, drops, dispersible powder or granule, syrup, elixir, ophthalmic preparation, food stuff, and any combination of forms thereof. In preferred embodiments, the forms are tablet, soft gelatin capsules, hard gelatin capsules, cream, gel, lotion and combination of forms thereof.

In another embodiment, the composition is formulated as a capsule. In some other embodiment, the composition is a solid dispersion. A solid dispersion is prepared by solvent evaporation, spray drying, lyophilization, hot melt extrusion, or hot melt dispersion.

In still another embodiment of the present disclosure, the composition can be administered to a subject through modes selected from a group comprising, but not limited to, topical administration, oral administration, intravenous administration, intra articular administration, intramuscular administration, and combinations thereof. In preferred embodiments, the mode of administration is oral, topical, intravenous intramuscular, and combinations thereof.

In an embodiment, the composition in addition the compound of formula-I and active ingredients comprises “Pharmaceutically acceptable carrier, diluent or excipient” which includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The present disclosure further relates to method of treating a subject suspected of having a disease and/or a condition.

In an embodiment, the method of treating a subject suspected having the condition, comprises administering therapeutically effective amount of the compound of formula-I to the subject suspected of having the condition.

In another embodiment, the method of treating a subject suspected having the condition, comprises administering therapeutically effective amount of the composition comprising the compound of formula-I, the active ingredient and an excipient, to the subject suspected of having the condition.

“Effective amount” or “therapeutically effective amount” refers to that amount of a compound or pharmaceutical composition of the invention which, when administered to a mammal, preferably a human, is sufficient to effect treatment, as defined below, of a disease, such as cancer, in the mammal, preferably a human. The amount of a compound or pharmaceutical composition of the invention which constitutes a “therapeutically effective amount” will vary depending on the compound or pharmaceutical composition, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

i. preventing the disease or condition from occurring in a mammal, in particular, when such mammal is predisposed to the condition but has not yet been diagnosed as having it;
ii. inhibiting the disease or condition, e.g. arresting its development;
iii. relieving or alleviating the disease or condition, e.g. causing regression of the disease or condition; or
iv. relieving or alleviating the symptoms resulting from the disease or condition, e.g., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

Diseases and/or conditions mentioned above includes but not limited to disease and condition mediated by protein kinases, including diseases and conditions mediated by aurora-2 kinase, c-kit and/or PDGFR-a. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

Additional embodiments and features of the present disclosure will be apparent to one of ordinary skill in art based upon description provided herein. The embodiments herein provide various features and advantageous details thereof in the description. Descriptions of well-known/conventional methods and techniques are omitted so as to not unnecessarily obscure the embodiments herein. The examples provided herein are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1: Preparation of the Compound of Formula-I-Diosgenin Polyethylene Glycol Succinate

About 130 L of Toluene was taken in 500 KL reactor with distillation facility. The toluene was heated to about 120° C., followed by adding about 10 g of Diosgenin and about 4 kg of Succinic anhydride added under stirring and complete dissolution was ensured. This reaction mixture was maintained at a temperature of 100° C. to 120° C. for about 16 hours to 18 hours. Once the reaction was completed, about 500 ml of Sulphuric acid and 70 kg of Polyethylene glycol was added and stirred for about 4 hours at a temperature of about 100° C. to 120° C. The reaction mixture was cooled, and toluene was removed under reduced pressure. About 250 L of Ethyl acetate was added and washed with about 25% brine solution for three times. The product was chilled and washed with Petroleum ether. The final product was dried under vacuum and the yield was about 26 kg of Diosgenin Polyethylene glycol succinate.

### Example 2: Preparation of the Compound of Formula-I-Diosgenin Polyethylene Glycol Succinate

About 8.5 g of Diosgenin was dissolved in about 30 ml of Toluene. About 1.31 g of Tritheylamine and about 2.5 g of Succinyl dichloride was added and stirred for about 1 hour at room temperature to obtain Reaction mixture-A. In a sperate vessel, about 2 g of Polyethylene glycol 1000 was dissolved in about 30 ml of Toluene and about 2.5 g Tritheylamine was added to obtain Reaction mixture-B. The reaction mixture B was added to reaction mixture-A slowly under stirring and the stirring was continued for about 1 hour at room temperature. The reaction mixture was cooled, and toluene was removed under reduced pressure. About 250 ml of Ethyl acetate was added and washed with about 25% brine solution for three times. The ethyl acetate was removed under reduced pressure. The product was dried under vacuum. The yield was about 22.1 g of Diosgenin Polyethylene glycol succinate.

### Example 3: Preparation of the Composition-Hot Melt Method

About 20 to 30 grams of Curcumin soluble compositions were prepared for formulations described in Table 1 by the following steps. Curcumin was used as the active ingredient in these examples as a non-limiting example. The Diosgenin polyethylene glycol succinate was heated at a temperature of about 70° C. to melt, then Curcumin was added with continuously stirring for about 2 hours to obtain a complex. The complex was homogenized at a speed of about 5000 rpm to 10000 rpm for about 5 minutes to 8 minutes, and then stirred with a magnetic stirring bar at about 50° C. for about 12 minutes to 15 minutes to obtain the composition. The composition was stored at a temperature of about 2° C. to 8° C.

TABLE 1
Composition comprising curcumin and Diosgenin polyethylene glycol
succinate.

| | Weight in g | | |
|---|---|---|---|
| | Composition | Composition | Composition |
| Component | I | II | III |
| Curcamin | 10 g | 5 g | 5 g |
| Diosgenin polyethylene | 10 g | 10 g | 15 g |
| glycol succinate | | | |
| Ratio of Curcumin and | 1:1 | 1:2 | 1:3 |
| Diosgenin Polyethylene | | | |
| glycol succinate | | | |

Solubility of the Compositions I, II and III are determined by adding water to the composition and control curcumin (without the Diosgenin polyethylene glycol succinate). The results are described in Table 2.

TABLE 2
Solubility of the composition

| | Component | Water in ml require to | |
|---|---|---|---|
| Component | weight | solubilize | Inference |
| Composition I | 100 mg | 1.8 ml | Soluble |
| Composition | 100 mg | 1.2 ml | Soluble |
| II | | | |
| Composition | 100 mg | 0.55 ml | Freely soluble |
| III | | | |
| Control | 100 mg | >1000 ml | Practically |
| | | | insoluble |

### Example 4: Preparation and Release of the Compositions I, II and III from Capsuels

The Composition I, Composition II and Composition III are encapsulated in Size “00” hard gelatin capsules and the average weight of each capsule is 500 mg.

The dissolution profile for the encapsulated Composition I, Composition II and Composition III are determined. The dissolution conditions and the release of Curcuminoids analyzed by HPLC.

### Dissolution Condition

Stirrer type: Basket
Stirrer RPM: 100
Dissolution Media: pH 6.8 phosphate buffer
Time: 1, 2, 4, 6, and 24 hours

### HPLC Condition

Column: C18 (4.6×25 cm)
Mobil phase: Mixture of citric acid in water (0.1%) and tetrahydrofuran (6:4).
Flow rate: 1 ml per min
Detection: 420 nm

### Release Profile Composition I, II and III

TABLE 3
Percentage release of curcumin from the capsule.

| Capsule | % Release Curcumin |
|---|---|
| Composition I | 60.2 |
| Composition II | 71.2 |
| Composition III | 85.5 |

### Example 5: Analysis of Active Ingredient Release from Phosphate Buffer

A collection of about 7 surfactants of various kinds were tested for their capability to release curcumin in pH 6.8 buffer. In a typical procedure, Curcumin is formulated with various commercially available surfactants by Solvent evaporation method. In this method, Curcumin 1 g dissolved in 10 ml of acetone and 3 g of the commercially available surfactants dissolved in 10 ml of acetone separately. The Curcumin in acetone solution is added to the surfactant solution/suspension slowly under stirring. Finally, the acetone is removed under reduced pressure. The resultant mixture is encapsulated in “00” hard gelatin capsules. The dissolution and HPLC conditions are given below.

### Dissolution Condition

Stirrer type: Basket
Stirrer RPM: 100
Dissolution Media: pH 6.8 phosphate buffer
Time: 1 hr

### HPLC Condition

Column: C18 (4.6×25 cm)
Mobil phase: Mixture of citric acid in water (0.1%) and tetrahydrofuran (6:4).
Flow rate: Bill per min
Detection: 420 nm

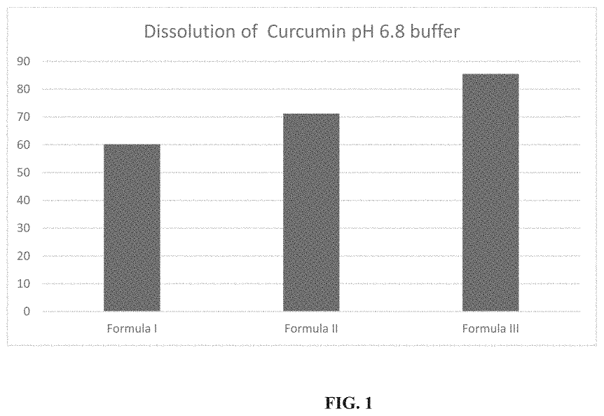

Curcumin with Diosgenin Polyethylene Glycol Succinate has the highest dissolution (solubility) (Table 3, FIG. 1) in pH 6.8 buffer.

TABLE 4
Comparison of release of Curcumin from a capsule comprising
commercially available surfactant and from the composition of
the present disclosure.

| | Curcumin Release in |
|---|---|
| Matrix | %, after 1 hr |
| Cur + HPMC | 4.61 |
| Curcumin 95% | 6.24 |
| Cur + Cavasol W7 HP | 9.09 |
| Cur + PEG 1000 | 19.83 |
| Cur + Vit E TPGS 1000 | 22.03 |
| Cur + Polaxamer 407 | 37.1 |
| Cur + Laktone RH 60 | 51.34 |
| Cur + Diosgenin | 87.24 |
| Polyethylene Glycol | |
| Succinate (DPGS) | |

Cur: Curcumin (Turmeric extract 95%), HPMC: Hydroxypropyl methyl cellulose, CAVASOL W7, HP: Hydroxypropyl-beta-cyclodextrin, PEG 1000: Polyethylene glycol 1000. Vit E TPGS 1000: Tocopherol polyethylene Glycol Succinate 1000, Kolliphor 407: Polaxamer 407, Laktone RH 60: Hydrogenated Castor Oil, DPGS: Diosgenin Polyethylene Glycol succinate.

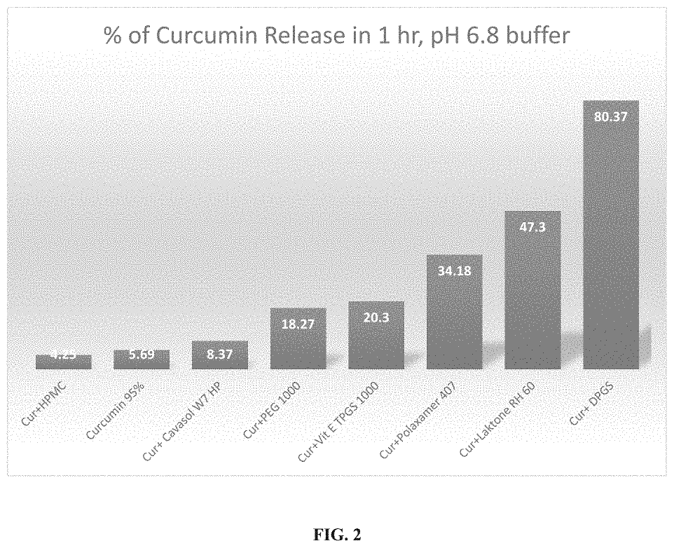

FIG. 2 illustrates the release of Curcumin from a capsule comprising commercially available surfactants and from the composition of the present disclosure, wherein it can be observed that the composition (CUR+SPEGE) of the present disclosure has significant (87.24%) release of curcumin when compared to combination of curcumin with commercially available surfactants, respectively.

### Example 6: Preparation of Curcumin Water

The composition of Curcumin water is given in the Table 5

TABLE 5

| S. | | |
|---|---|---|
| No. | Ingredient | Composition |
| 1 | Curcumin:Diosgenin Polyethylene | 425 mg |
| | Glycol succinate (1:3) complex | |
| 2 | Lemon flavour | 20 mg |
| 3 | Ascorbic acid | 300 mg |
| 4 | Tartaric acid | 100 mg |
| 4 | Water | Q.S |

Q.S: Quantity sufficient

Curcumin: Diosgenin Polyethylene Glycol succinate (1:3) complex is prepared by hot melt extrusion method. In a typical procedure, Curcumin and Diosgenin Polyethylene Glycol succinate in the ratio of 1:3 was taken and subjected to hot melt extrusion at 180° C. In sufficient quantity of water, the Curcumin: Diosgenin Polyethylene Glycol succinate (1:3) complex, Ascorbic acid, Tartaric acid and lemon flavor were dissolved and filtered.

### Example 7: Preparation of Forksolin Water-Solvent Dissolution Method

The composition of Forskolin water is given in the Table 6

TABLE 6

| S. | | |
|---|---|---|
| No. | Ingredient | Composition |
| 1 | Forskolin and Diosgenin Polyethylene | 100 mg |
| | Glycol succinate complex (1:3) | |
| 2 | Maleic acid | 100 mg |
| 3 | Tartaric acid | 40 mg |
| 4 | Strawberry flavour | 20 mg |
| 5 | Water | Q.S |

Q.S: Quantity Sufficient

Forskolin and Diosgenin Polyethylene Glycol Succinate complex (1:3) is prepared by solvent evaporation method. In a typical procedure, Forskolin 1 part and Diosgenin Polyethylene glycol succinate 3 part were dissolved in Isopropyl alcohol separately. The Forskolin solution is added to Diosgenin Polyethylene Glycol succinate solution slowly under stirring. The solvent Isopropyl alcohol is evaporated under vacuum. The Forskolin and Diosgenin Polyethylene glycol succinate complex, Maleic acid, Tartaric acid and Strawberry flavor were dissolved in 200 ml water and Filtered.

### Example 8: Preparation of Tobacco De-Addiction Hard Candies

The composition of Tobacco de-addiction hard candies is given in the Table 7

TABLE 7

| S. | | |
|---|---|---|
| No. | Ingredient | Composition |
| 1 | Curcumin and Diosgenin Polyethylene | 100 mg |
| | glycol succinate complex (1:3) | |
| 2 | Licorice extract | 100 mg |
| 3 | Clove oil | 10 mg |
| 4 | Piper nigrum extract | 5 mg |
| 5 | Zanthoxylum rhetsa extract | 5 mg |
| 5 | Sugar | Q.S |
| 6 | Corn syrup | Q.S |
| 7 | Water | Q.S |

Q.S: Quantity Sufficient

Sugar and Corn syrup were added to water and heated till boiling. The composition comprising Curcumin and Diosgenin Polyethylene succinate, Licorice extract, clove oil, piper nigrum extract and Zanthoxylum rhetsa extract were added under stirring. The mixture was poured in a tray, cooled and cut into bite size pieces.

### Example 9: Preparation of Antacid Effercescent Granules

The composition of Antacid effervescent is given in the Table 8

TABLE 8

| S. | | |
|---|---|---|
| No. | Ingredient | Composition |
| 1 | Curcumin and Diosgenin Polyethylene | 100 mg |
| | Glycol succinate complex (1:3) | |
| 2 | Liquorice extract | 100 mg |
| 3 | Cucumber extract | 10 mg |
| 4 | Lemon flavour | 5 mg |
| 5 | Citric acid | 100 mg |
| 6 | Sodium bicarbonate | 100 mg |
| 7 | Water | Q.S |

Q.S: Quantity Sufficient

The composition comprising Curcumin and Diosgenin Polyethylene glycol succinate was heated and Licorice extract, Cucumber extract and lemon flavor were added under stirring condition to obtain a mixture. The obtained mixture was added to Citric acid and Sodium bicarbonate mixture, slowly and it was subjected granulation.

### Example 10: Preparation of Stable Water Soluble Caroteinoids

The composition of Stable carotenoids formulation is given in the Table 9

### For 100 g Carotenoids 10% Beadlets

TABLE 9

| S. | | |
|---|---|---|
| No. | Ingredient | Composition |
| 1 | Carotenoids (Lutein/Zeaxanthin 5:1) 80% | 12.5 g |
| 2 | Diosgenin Polyethylene glycol succinate | 12.5 g |
| 3 | Sodium alginate | 2.5 g |
| 4 | Ethyl cellulose | 2.5 g |
| 5 | Sugar/Starch Beads | 50 g |
| 6 | Ethanol | Q.S |

For Barrier coating

| | | |
|---|---|---|
| 7 | Diosgenin Polyethylene glycol succinate | 20 g |
| 8 | Ethanol | Q.S |

Q.S: Quantity Sufficient

Carotenoids, Diosgenin Polyethylene glycol succinate, Sodium Alginate and Ethyl cellulose were dissolved in suitable quantity of Ethanol to obtain a solution. The solution was sprayed on to a coating pan containing Sugar/Starch beads. The barrier coating is prepared by dissolving 20 g of Diosgenin Polyethylene Glycol Succinate in Ethanol and Sprayed as a barrier coating.

### Example 11: Preparation of Depigmentation Cream

The composition of depigmentation cream is given in Table 10

TABLE 10

| S. No | Ingredients | % w/w |
|---|---|---|
| A | Isopropyl myristate | 4.0 |
| | Caprylic/Capric triglycerides | 3.0 |
| | Lanolin alcohol | 1.5 |
| | Emulsifying wax NF | 3.5 |
| B | CETETH-20 (Cetomacrogol-1000) | 1.5 |
| | Glycerin | 2.0 |
| | Tetrasodium EDTA | 0.02 |
| | Imidurea | 0.15 |
| | Sodium methylparaben | 0.2 |
| | Sodium propylparaben | 0.02 |
| | Demineralized water | QS |
| C | Ellagic acid | 1.0 |
| | Licorice extract | 2.0 |
| | Diosgenin Polyethylene glycol Succinate | 6.0 |
| D | Novemer EC-1 | 3.0 |
| | Triethanolamine | QS |

Q.S: Quantity Sufficient

### Procedure of Preparing the Depigmentation Cream

Part A ingredients were combined and heated to a temperature of about 70° C. to 75° C.;
Part B ingredients were combined in a separate vessel and heated to a temperature of about 70° C. to 75° C.;
Part C-The Diosgenin Polyethylene glycol Succinate was heated to a temperature of about 70° C. to 75° C. and Ellagic acid and Licorice extract were added;
Part A is added to Part B with continuous agitation and then Part C was added; and
Finally, Part D was added and mixed to form a homogenous mixture.

From the foregoing it will be appreciated that, although specific embodiments of the invention have been described herein for purposes of illustration, various modifications may be made without deviating from the spirit and scope of the invention.

We claim:

1. A compound of Formula-I

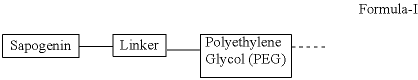

2. The compound as claimed in claim 1, wherein the Sapogenin is selected from a group comprising Diosgenin, Solasodin, Tomatidine, 11-Ketotigogenin, Hecogenin, Laxogenin, Smilagenin, Sarsasapogenin, Tigogenin, Epismilagenin, Yamogenin and any combination thereof.

3. The compound as claimed in claim 1, wherein the linker is carboxylic acid, dicarboxylic acid or derivative thereof selected from a group comprising Succinate, adipate, glutarate, pimelate malonate and Sebecate.

4. The compound as claimed in claim 1, wherein the PEG is selected from a group comprising PEG 200, PEG 400, PEG 600, PEG 800, PEG 1000, PEG 2000 and any combination thereof.

5. The compound as claimed in claim 1, Wherein the compound is selected from a group Diosgenin Polyethylene glycol succinate, Diosgenin Polyethylene Glycol Adipate, Diosgenin Polyethylene Glycol Glutarate, Diosgenin Polyethylene Glycol Pimelate, Diosgenin Polyethylene Glycol Malonate, Diosgenin Polyethylene Glycol Sebecate, Tomatidine Polyethylene glycol succinate, Tomatidine Polyethylene Glycol Adipate, Tomatidine Polyethylene Glycol Glutarate, Tomatidine Polyethylene Glycol Pimelate, Tomatidine Polyethylene Glycol Malonate, Tomatidine Polyethylene Glycol Sebecate, 11-Ketotigogenin Polyethylene glycol succinate, 11-Ketotigogenin Polyethylene Glycol Adipate, 11-Ketotigogenin Polyethylene Glycol Glutarate, 11-Ketotigogenin Polyethylene Glycol Pimelate, 11-Ketotigogenin Polyethylene Glycol Malonate, 11-Ketotigogenin Polyethylene Glycol Sebecate, Hecogenin Polyethylene glycol succinate, Hecogenin Polyethylene Glycol Adipate, Hecogenin Polyethylene Glycol Glutarate, Hecogenin Polyethylene Glycol Pimelate, Hecogenin Polyethylene Glycol Pimelate, Hecogenin Polyethylene Glycol Malonate, Hecogenin Polyethylene Glycol Sebecate, Laxogenin Polyethylene glycol succinate, Laxogenin Polyethylene Glycol Adipate, Laxogenin Polyethylene Glycol Glutarate, Laxogenin Polyethylene Glycol Pimelate, Laxogenin Polyethylene Glycol Malonate, Laxogenin Polyethylene Glycol Sebecate, Smilagenin Polyethylene glycol succinate, Smilagenin Polyethylene Glycol Adipate, Smilagenin Polyethylene Glycol Glutarate, Smilagenin polyethylene Glycol Pimelate, Smilagenin Polyethylene Glycol Malonate, Sarsasapogenin Polyethylene glycol succinate, Sarsasapogenin Polyethylene Glycol Adipate, Sarsasapogenin Polyethylene Glycol Glutarate, Sarsasapogenin Polyethylene Glycol Pimelate, Sarsasapogenin Polyethylene Glycol Malonate Sarsasapogenin Polyethylene Glycol Sebecate, Tigogenin Polyethylene glycol succinate, Tigogenin Polyethylene Glycol Adipate, Tigogenin Polyethylene Glycol Glutarate, Tigogenin Polyethylene Glycol Pimelate, Tigogenin Polyethylene Glycol Malonate, Tigogenin Polyethylene Glycol Sebecate, Epismilagenin Polyethylene Glycol succinate, Epismilagenin Polyethylene Glycol Adipate, Epismilagenin Polyethylene Glycol Glutarate, Epismilagenin Polyethylene Glycol Pimelate, Epismilagenin Polyethylene Glycol Malonate, Epismilagenin Polyethylene Glycol Sebecate, Yamogenin Polyethylene Glycol succinate, Yamogenin Polyethylene Glycol Adipate, Yamogenin Polyethylene Glycol Glutarate, Yamogenin Polyethylene Glycol Pimelate, Yamogenin Polyethylene Glycol Malonate and Yamogenin Polyethylene Glycol Sebecate.

6. A method for preparing the compound as claimed in claim 1, said method comprising—
a) mixing the sapogenin, the carboxylic acid/dicarboxylic acid/derivative thereof, the PEG and solvent-A to obtain a mixture;
b) cooling the mixture, followed by removing solvent-A;
c) adding solvent-B, followed by washing with solution; and
d) cooling the mixture of step c), followed by washing with Solvent-C and drying to obtain the compound.

7. The method as claimed in claim 6, wherein the mixing of step a) comprises—
a) heating solvent-A, followed by adding sapogenin and the carboxylic acid/dicarboxylic acid/derivative and continuing heating; and
b) adding an acid and PEG, followed by heating to obtain a mixture;

8. The method as claimed in claim 6, wherein the mixing of step a) optionally comprises—
a) Mixing sapogenin and solvent-A, followed by adding a base and carboxylic acid/dicarboxylic acid/derivative to obtain mixture-A;
b) Mixing PEG, solvent-A and base to obtain mixture-B; and
c) Mixing the mixture-A and the mixture-B.

9. The method as claimed in claim 6, wherein the solvent-A is selected from a group comprising Toluene, N N Dimethylformamide, Xylene and combinations thereof; wherein the solvent-B is selected from a group comprising ethyl acetate, Isopropyl acetate, Isobutyl acetate and combinations thereof; wherein the solvent-C is selected from a group comprising petroleum ether, diethyl ether, methyl t-butyl ether and combinations thereof; and wherein the solution is brine solution.

10. The method as claimed in claim 6, wherein the mixture in step b) is cooled to a temperature ranging from about 5° C. to 15° C.; and wherein the mixture of step d) is cooled to a temperature ranging from about 0° C. to 5° C.

11. The method as claimed in claim 7, wherein the solvent-A is heated to a temperature ranging from about 80° C. to 90° C.; and wherein the heating is continued to a temperature ranging from about 100° C. to 120° C. for a duration ranging from about 16 hours to 18 hours.

12. The method as claimed in claim 7, wherein the heating in step b) is carried out for a duration ranging from about 100° C. to 120° C. for a duration ranging from about 2 hours to 8 hours.

13. A composition comprising the compound as claimed in claim 1 and active ingredient selected from a group comprising active pharmaceutical ingredient, dietary ingredient and cosmetic ingredient, optionally along with excipient.

14. The composition as claimed in claim 1, wherein ratio of the active ingredient to the compound is ranging from about 1:1 to 1:10.

15. A method of preparing the composition as claimed in claim 13, said method comprises—
Mixing the compound as claimed in claim 1 and the active ingredient to obtain a mixture; and
Homogenizing the mixture to obtain the composition.

16. The method as claimed in claim 15, wherein mixing is carried out a temperature ranging from about 50° C. to 100° C. for a duration ranging from about 1 hour to 3 hours; and wherein the homogenizing is carried out at a speed ranging from about 5000 rpm to 10000 rpm for a duration ranging from about 5 minutes to 10 minutes.

17. The method as claimed in claim 15, wherein the method additionally comprises stirring the homogenized mixture at a temperature ranging from about 25° C. to 50° C. for a duration ranging from about 10 minutes to 15 minutes.
